(19) United States
(12) Patent Application Publication
(10) Pub. No.: US 2007/0249562 A1
(43) Pub. Date: Oct. 25, 2007

(54) TREATMENT OF ATRIAL FIBRILLATION

(76) Inventor: Albert D. Friesen, Winnipeg (CA)

Correspondence Address: MERCHANT & GOULD PC, P.O. BOX 2903, MINNEAPOLIS, MN, 55402-0903, US

(21) Appl. No.: 11/739,920

(22) Filed: Apr. 25, 2007

Related U.S. Application Data
(60) Provisional application No. 60/745,581, filed on Apr. 25, 2006.

(51) Int. Cl. A61K 31/675; A61K 31/4415

(52) U.S. Cl. 514/89; 514/350

(57) ABSTRACT

The invention includes a method of treating atrial fibrillation in a mammal that includes administering a therapeutically effective amount of at least one therapeutic compound Compounds suitable for use in the methods of the invention include pyridoxal-5′-phosphate, pyridoxic acid, pyridoxamine, pyridoxal, 3-acylated pyridoxal analogues, pharmaceutically acceptable acid addition salts thereof, and mixtures thereof.

### CROSS-REFERENCE TO RELATED APPLICATION

This application claims the benefit under 35 U.S.C. § 119(e) of United States Provisional Patent Application Ser. No. 60/745,581, filed Apr. 25, 2006, the entire disclosure of which is hereby incorporated by reference.

### FIELD OF THE INVENTION

This invention relates to a method of treating atrial fibrillation,

### BACKGROUND

Atrial fibrillation (AF) affects more the 2.2 million people in the United States. The prevalence increases with age and tends to occur more in males than females. Approximately 4% of people over the age of 60 have experienced an episode of AF.

The atria are the two upper chambers of the heart. During AF, the atria do not contract normally but rather quiver in a rapid, chaotic fashion. Clinically it is diagnosed by irregular rhythm and an absence of P waves on an ECG. Also, the ECG of a patient with AF will usually show a narrow QRS complex, although it may be wide if abnormal conduction or partial or full interruption of electrical conduction in the bundle blocks is present.

AF can occur in healthy people, but more often is associated with an underlying condition such as coronary heart disease, hypertension, valvular heart disease, or rheumatic heart disease. AF may also develop after cardiac or pulmonary surgery. AF is harmful since without proper contraction, the blood is not effectively moved out of the atria. This may result in atrial blood pooling and blood clot formation. Blood clots may leave the heart and block vessels in the brain which may lead to stroke.

Myocardial infarction is necrosis of a region of the myocardium caused by an interruption in the supply of blood to the heart, usually as a result of occlusion of a coronary artery.

Coronary artery bypass grafting (CABG) is performed to bypass blockages or obstructions of the coronary arteries. This results in a better quality of life in specific subgroups of patients with obstructive coronary artery disease. Due to the high incidence of coronary artery disease worldwide, as well as the effectiveness of this surgical procedure, CABG surgery makes up one of the top ten most frequently performed procedures in North America and Europe. According to the European Heart Survey and the National Registries of Cardiovascular Diseases and Patient Management, the total volume of bypass surgery was over 280,000 in the 15 European Union countries in the year 2000. In the United States it is estimated that over 700,000 CABG procedures are performed per year.

Despite the benefits of CABG surgery, patients undergoing these procedures may also suffer serious adverse outcomes including operative mortality, myocardial infarction, unstable angina, ventricular failure, life-threatening arrhythmia, renal insufficiency, and stroke. Some of the proposed causes of cardiovascular morbidity and mortality after CABG include perioperative ischemia, inadequate myocardial protection and reperfusion injury. The impact of these serious complications is significant. Incidence rates of death and myocardial infarction following CABG surgery range from 5% to 12% depending on risk status. Results from large clinical trials have recently demonstrated the importance of neurologic deficits as a problematic outcome of CABG. These deficits include impairment of memory, psychomotor, visuospatial, attention, and language abilities as measured by neuropsychological testing as well as the sensori-motor abnormalities associated with stroke.

### SUMMARY OF THE INVENTION

The invention includes a method of treating atrial fibrillation in a mammal that includes administering a therapeutically effective amount of at least one therapeutic compound. Compounds suitable for use in the methods of the invention include pyridoxal-5′-phosphate, pyridoxic acid, pyridoxamine, pyridoxal, 3-acylated pyridoxal analogues, pharmaceutically acceptable acid addition salts thereof, and mixtures thereof. In an embodiment, the therapeutic compound is administered following a surgery. In another embodiment, the therapeutic compound is pyridoxal-5′-phosphate.

### DETAILED DESCRIPTION

The invention includes a method of treating atrial fibrillation in a mammal that includes administering a therapeutically effective amount of at least one therapeutic compound. Compounds suitable for use in the methods of the invention include pyridoxal-5′-phosphate, pyridoxic acid, pyridoxamine, pyridoxal, 3-acylated pyridoxal analogues, pharmaceutically acceptable acid addition salts thereof, and mixtures thereof.

### Therapeutic Compounds Suitable for Use in Methods of the Invention

Methods of the invention include administration of a therapeutically effective amount of a compound including pyridoxal-5′-phosphate (P5P), pyridoxal, pyridoxamine, pyridoxic acid, 3-acylated analogues of pyridoxal, 3-acylated analogue of pyridoxal-4,5-aminal, pharmaceutically acceptable acid salts, and mixtures thereof.

Pyridoxal-5′-phosphate, an end product of vitamin B₆ metabolism, plays a vital role in mammalian health. Vitamin B₆ typically refers to pyridoxine, which is chemically known as 2-methyl-3-hydroxy-4,5-di(hydroxymethyl)pyridine and is represented by formula I: 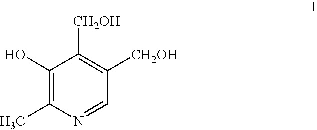
Yet two additional compounds, pyridoxal (formula II) 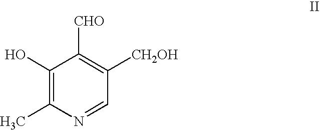
and pyridoxamine (formula III) 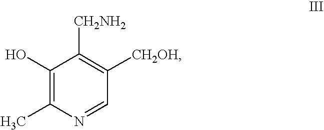
are also referred to as vitamin B₆. All three compounds serve as precursors to pyridoxal-5′-phosphate, also known as 3-hydroxy-2-methyl-5-[(phosphonooxy) methyl]-4-pyridine-carboxaldehyde and is represented by formula IV: 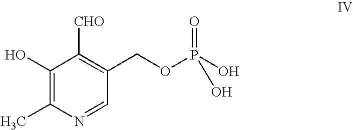
Pyridoxal-5′-phosphate is a metabolite of vitamin B₆ inside cells and in blood plasma. Mammals cannot synthesize pyridoxal-5′-phosphate de novo and must rely on dietary sources of the precursors pyridoxine, pyridoxal, and pyridoxamine, which are metabolized to pyridoxal-5′-phosphate. For instance, mammals produce pyridoxal-5′-phosphate by phosphorylating pyridoxine by action of pyridoxal kinase and then oxidizing the phosphorylated product.

Pyridoxal-5′-phosphate is a regulator of biological processes and a cofactor in many enzymatic reactions. It is hypothesized that pyridoxal-5′-phosphate might prevent or reduce tissue damage during ischemia and reperfusion episodes by blocking calcium influx. The biological role of pyridoxal-5′-phosphate is believed to also include acting as a coenzyme and as an antagonist. Pyridoxal-5′-phosphate is a coenzyme at the glycogen phosphorylase level (glycogenolysis) and at the transamination level in the malate aspartate shuttle (glycolysis and glycogenolysis). A recent evaluation demonstrated that pyridoxal-5′-phosphate inhibits adenosine triphosphate (ATP) induced calcium ion influx into cells. Results suggest that this action is due to an inhibition of purinergic receptors known as P_{2X} purinoceptors.

Pyridoxal-5′-phosphate can be chemically synthesized in a number of ways, for example, by the action of ATP on pyridoxal, by the action of phosphorus oxychloride on pyridoxal in aqueous solution, and by phosphorylation of pyridoxamine with concentrated phosphoric acid followed by oxidation.

Therapeutic compounds include esters of pyridoxic acid and pyridoxic acid-4,5-lactone.

Therapeutic compounds also include any one or more of the 3-acylated analogues of pyridoxal represented by formula V:

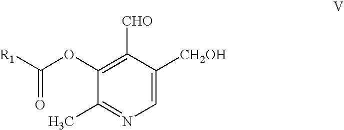

wherein

R₁ is alkyl,
alkenyl,
   in which alkyl or alkenyl
      can be interrupted by nitrogen, oxygen, or sulfur, and
      can be unsubstituted or substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy;
aryl,
   in which aryl can be substituted by alkyl, alkoxy, amino, hydroxy, halo, nitro, or alkanoyloxy;
aryloxy;
arylthio; or
aralkyl,
or a pharmaceutically acceptable acid addition salt thereof.

Therapeutic compounds also include any one or more of the 3-acylated analogues of pyridoxal-4,5-aminal represented by formula VI:

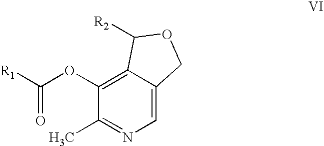

wherein

R₁ is alkyl,
alkenyl,
   in which alkyl or alkenyl
      can be interrupted by nitrogen, oxygen, or sulfur, and
      can be unsubstituted or substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy;
aryl,
   in which aryl can be substituted by alkyl, alkoxy, amino, hydroxy, halo, nitro, or alkanoyloxy;
aryloxy;
arylthio; or
aralkyl; and

R₂ is of the formula 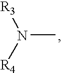
wherein R₃ and R₄ are each independently alkyl, alkenyl, cycloalkyl, aryl, or, when R₃ and R₄ are taken together to form a ring with the nitrogen atom, which may optionally be interrupted by a heteroatom,

or a pharmaceutically acceptable acid addition salt thereof.

The term “alkyl” includes a straight or branched saturated aliphatic hydrocarbon radicals, such as, for example, methyl, ethyl, propyl, isopropyl (1-methylethyl), 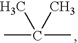
butyl, tert-butyl (1,1-dimethylethyl), and the like. In one embodiment, alkyl has from 1 to 8 carbon atoms. In another embodiment, alkyl has from 1 to 6 carbon atoms. In another embodiment, alkyl has from 1 to 4 carbon atoms. In one embodiment, alkyl has 1 carbon. The alkyl group may optionally be substituted with one or more substituents such as fluorine, chlorine, alkoxy groups having from 1 to 8 carbon atoms (e.g., methoxy or ethoxy), or amido groups having from 1 to 8 carbon atoms, such as acetamido. These substituents may themselves be substituted with one or more functional groups such as hydroxy groups, carboxy groups, acetoxy groups, or halogens.

The term “alkenyl” group includes an unsaturated aliphatic hydrocarbon chain having from 2 to 8 carbon atoms, such as, for example, ethenyl, 1-propenyl, 2-propenyl, 1-butenyl, 2-methyl-1-propenyl, and the like.

The above alkyl or alkenyl groups may optionally be interrupted in the chain by a heteroatom, such as, for example, a nitrogen, sulfur, or oxygen atom, forming an alkylaminoalkyl or alkoxyalkyl group, for example, methylaminoethyl or methoxymethyl, and the like. The above alkyl or alkenyl can also optionally be substituted at the terminal carbon by hydroxy, alkoxy, alkanoyloxyaryl, alkanoyloxy, alkoxyalkanoyl, alkoxycarbonyl, or dialkylcarbamoyloxy.

The term “alkoxy” group includes an alkyl group as defined above joined to an oxygen atom having preferably from 1 to 4 carbon atoms in a straight or branched chain, such as, for example, methoxy, ethoxy, propoxy, isopropoxy (1-methylethoxy), butoxy, tert-butoxy (1,1-dimethylethoxy), and the like.

The term “dialkylamino” group includes two alkyl groups as defined above joined to a nitrogen atom, in which the alkyl group has preferably 1 to 4 carbon atoms, such as, for example, dimethylamino, diethylamino, methylethylamino, methylpropylamino, diethylamino, and the like.

As used herein “aryl” means a mono- or poly-nuclear aromatic hydrocarbon radical. Examples of “aryl” groups include, but are not limited to aromatic hydrocarbons such as a phenyl group or a naphthyl group. The aromatic group may optionally be substituted with one or more substituents such as fluorine, chlorine, alkyl groups having from 1 to 8 carbon atoms (e.g., methyl or ethyl), alkoxy groups having from 1 to 8 carbon atoms (e.g., methoxy or ethoxy), alkoxyalkyl groups having from 1 to 8 carbon atoms and one or more oxygen atoms, or amido groups having from 1 to 8 carbon atoms, such as acetamido. These substituents may themselves be substituted with one or more functional groups such as hydroxy groups, carboxy groups, acetoxy groups, or halogens.

In one embodiment, aryl is a phenyl group or a naphthyl group that is either unsubstituted or substituted.

In another embodiment, aryl is a heteroaryl in which one or more of the carbon atoms of an aromatic hydrocarbon are substituted with a nitrogen, sulfur, or oxygen. Examples of a “heteroaryl” include, but are not limited to pyridine, pyrimidine, pyran, dioxin, oxazine, and oxathiazine. Likewise, the heteroaryl may optionally be substituted with functional groups such as hydroxy groups, carboxy groups, halogens, and amino groups.

The term “alkanoyloxy” includes a group of the formula 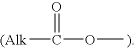
Examples of alkanoyloxy include methanoyloxy, ethanoyloxy, propanoyloxy, and the like. Examples of alkyl substituted at the terminal carbon by alkanoyloxy include 1-ethanoyloxy-1-methylethyl, propanoyloxy-1-methylethyl, and the like.

The term “alkanoyloxyaryl” includes a group of the formula 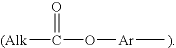
Examples of alkanoyloxyaryl include methanoyloxyphenyl, ethanoyloxyphenyl, propanoyloxyphenyl, and the like.

The term “aryl” refers to unsaturated aromatic carbocyclic radicals having a single ring, such as phenyl, or multiple condensed rings, such as naphthyl or anthryl. The term “aryl” also includes substituted aryl comprising aryl substituted on a ring by, for example, C₁₋₄ alkyl, C₁₋₄ alkoxy, amino, hydroxy, phenyl, nitro, halo, carboxyalkyl or alkanoyloxy. Aryl groups include, for example, phenyl, naphthyl, anthryl, biphenyl, methoxyphenyl, halophenyl, and the like.

The term “aryloxy” (i.e. aryl-O—) includes aryl having an oxygen atom bonded to an aromatic ring, such as, for example, phenoxy and naphthoxy.

The term “arylthio” (i.e. aryl-S—) includes aryl having a sulfur atom bonded to an aromatic ring, such as, for example, phenylthio, and naphthylthio.

The term “aralkyl” refers to an aryl radical defined as above substituted with an alkyl radical as defined above (e.g. aryl-alkyl-). Aralkyl groups include, for example, phenethyl, benzyl, and naphthylmethyl.

The term “alkoxyalkanoyl” includes a group of the formula 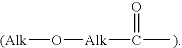
Examples of alkoxyalkanoyl include (2-acetoxy-2-methyl)propanyl, 3-ethoxy-3-propanoyl, 3-methoxy-2-propanoyl, and the like.

The term “alkoxycarbonyl” includes a group of the formula 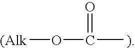
Examples of alkoxycarbonyl include methoxycarbonyl, ethoxycarbonyl, propoxycarbonyl, and the like.

The term “dialkylcarbamoyloxy” includes a group of the formula 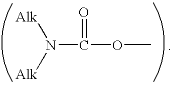
Examples of dialkylcarbamoyloxy include dimethylamino-methanoyloxy, 1-ethyl-1-methylaminomethanoyloxy, and the like. Examples of alkyl substituted at the terminal carbon by alkanoyloxy include dimethylamino-1-methylethyl, 1-ethyl-1-methylaminomethanoyloxy-1-methylethyl, and the like.

The term “halo” includes bromo, chloro, and fluoro.

A group of the formula: 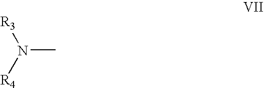
is derived from a secondary amine R₁R₂NH, in which R₁ and R₂ are each independently alkyl, alkenyl, cycloalkyl, aryl, or, when R₁ and R₂ are taken together, may form a ring with the nitrogen atom and which may optionally be interrupted by a heteroatom, such as, for example, a nitrogen, sulfur, or oxygen atom. The terns “alkyl,” “alkenyl,” and “aryl” are used as defined above in forming secondary amino groups such as, for example, dimethylamino, methylethyl amino, diethylamino, dialkyl amino, phenylmethylamino, diphenylamino, and the like.

Examples of 3-acylated analogues of pyridoxal include, but are not limited to, 2-methyl-3-toluoyloxy-4-formyl-5-hydroxymethylpyridine and 2-methyl-β-naphthoyloxy-4-formyl-5-hydroxymethylpyridine. Examples of compounds of formula V and methods of synthesizing those compounds are described in U.S. Pat. No. 6,890,943, the disclosure of which is incorporated herein by reference.

Pharmaceutically acceptable salts include acid addition salts derived from nontoxic inorganic acids such as hydrochloric, nitric, phosphoric, sulfuric, hydrobromic, hydriodic, hydrofluoric, phosphorus, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate. trifluoroacetate, propionate, caprylate, isobutyrate, oxalate, malonate, succinate, suberate, sebacate, fumarate, maleate, mandelate, benizoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glutamine, etc. (see, e.g., Berge et al., J. Pharmaceutical Science, 66: 1-19 (1977)).

The salts of the basic compounds are prepared by contacting the flee base form with a sufficient amount of a desired acid to produce the salt in the conventional manner. The free base form can be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable salts of the compounds include metals such as alkali and alkaline earth metals. Examples of metals used as cations are sodium, potassium, magnesium, calcium, and the like. Also included are heavy metal salts such as for example silver, zinc, cobalt, and cerium.

Pharmaceutical Compositions

A therapeutic compound as defined above can be formulated into a pharmaceutical composition for use in methods of the invention. A pharmaceutical composition is suitable for treating atrial fibrillation.

Although it is possible for compounds of the invention to be administered alone in a unit dosage form, the compounds are typically administered in admixture with a carrier as a pharmaceutical composition to provide a unit dosage form. The invention provides pharmaceutical compositions containing at least one compound of the invention. A pharmaceutical composition comprises a pharmaceutically acceptable carrier in combination with a compound of the invention or a pharmaceutically acceptable salt of a compound of the invention.

A pharmaceutically acceptable carrier includes, but is not limited to, physiological saline, ringers, phosphate-buffered saline, and other carriers known in the art. Pharmaceutical compositions can also include additives such as, for example, stabilizers, antioxidants, colorants, excipients, binders, thickeners, dispersing agents, readsorpotion enhancers, buffers, surfactants, preservatives, emulsifiers, isotonizing agents, and diluents. Pharmaceutically acceptable carriers and additives are chosen such that side effects from the pharmaceutical compound are minimized and the performance of the compound is not canceled or inhibited to such an extent that treatment is ineffective.

Methods of preparing pharmaceutical compositions containing a pharmaceutically acceptable carrier in combination with a therapeutic compound of the invention or a pharmaceutically acceptable acid addition salt of a compound of the invention are well known. All methods can include the step of bringing a compound of the invention in association with a carrier and/or additives. Formulations generally are prepared by uniformly and intimately bringing the compound of the invention into association with a liquid carrier or a finely divided solid carrier or both, and then, if necessary, shaping the product into the desired unit dosage forms.

Generally, a solution of a therapeutic compound, for example pyridoxal-5′-phosphate, may be prepared by simply mixing pyridoxal-5′-phosphate with a pharmaceutically acceptable solution, for example, buffered aqueous saline solution at a neutral or alkaline pH (because pyridoxal-5′-phosphate is essentially insoluble in water, alcohol, and ether), at a temperature of at least room temperature and under sterile conditions. Preferably, the pyridoxal-5′-phosphate solution is prepared immediately prior to administration to the mammal. However, if the pyridoxal-5′-phosphate solution is prepared at a time more than immediately prior to the administration to the mammal, the prepared solution should be stored under sterile, refrigerated conditions. Furthermore, because pyridoxal-5′-phosphate is light sensitive, the pyridoxal-5′-phosphate solution should be stored in containers suitable for protecting the pyridoxal-5′-phosphate solution from the light, such as amber-colored vials or bottles.

For oral administration as a tablet or capsule, compositions can be prepared according to techniques well known in the art of pharmaceutical formulation. Compositions can contain microcrystalline cellulose for imparting bulk, alginic acid, or sodium alginate as a suspending agent, methylcellulose as a viscosity enhancer, and sweeteners or flavoring agents. As immediate release tablets, compositions can contain microcrystalline cellulose, starch, magnesium stearate and lactose or other excipients, binders, extenders, disintegrants, diluents and lubricants known in the art.

For administration by inhalation or aerosol, compositions can be prepared according to techniques well known in the art of pharmaceutical formulation. Compositions can be prepared as solutions in saline, using benzyl alcohol or other suitable preservatives, absorption promoters to enhance bioavailability, fluorocarbons or other solubilizing or dispersing agents known in the art.

For administration as injectable solutions or suspensions, compositions can be formulated according to techniques well-known in the art, using suitable dispersing or wetting and suspending agents, such as sterile oils, including synthetic mono- or di-glycerides, and fatty acids, including oleic acid.

For rectal administration as suppositories, compositions can be prepared by mixing with a suitable non-irritating excipient, such as cocoa butter, synthetic glyceride esters or polyethylene glycols, which are solid at ambient temperatures, but liquefy or dissolve in the rectal cavity to release the drug.

Method of Treatment Using Compounds of the Invention

In another aspect of the invention, methods are provided for the treatment of atrial fibrillation.

As used herein, the terms “treatment” and “treating” include inhibiting, alleviating, and healing atrial fibrillation or symptoms thereof. Treatment can be carried out by administering a therapeutically effective amount of at least one compound of the invention. A “therapeutically effective amount” as used herein includes a prophylactic amount, for example, an amount effective for alleviating or healing the above mentioned diseases or symptoms thereof.

A physician or veterinarian of ordinary skill readily determines a mammalian subject who is exhibiting symptoms of atrial fibrillation. Regardless of the route of administration selected, a compound of the invention or a pharmaceutically acceptable acid addition salt of a compound of the invention can be formulated into pharmaceutically acceptable unit dosage forms by conventional methods known in the pharmaceutical art. An effective but nontoxic quantity of the compound is employed in treatment. Compounds can be administered in enteral unit dosage forms, such as, for example, tablets, sustained-release tablets, enteric coated tablets, capsules, sustained-release capsules, enteric coated capsules, pills, powders, granules, solutions, and the like. Compounds can also be administered parenterally, such as, for example, subcutaneously, intramuscularly, intradermally, intramammarally, intravenously, and by other administrative methods known in the art. A method of the invention can also include delivering a compound via a medical device, wherein the compound is reversibly bound to at least one surface of the medical device. For example, a drug eluting intravascular stent can have at least one surface coated with pyridoxal-5′-phosphate. Further, an intravascular stent can be coated with a physiologically compatible matrix adapted for delayed release of a compound of a pharmaceutically acceptable salt thereof.

An ordinarily skilled physician or veterinarian will readily determine and prescribe a therapeutically effective amount of at least one compound to treat atrial fibrillation. The physician or veterinarian could employ relatively low dosages at first, subsequently increasing the dose until a maximum response is obtained. Typically, the severity of atrial fibrillation, the compound to be administered, the route of administration, and the characteristics of the mammal to be treated, for example, age, sex, and weight, are considered in determining the effective amount to administer. Administering a therapeutic amount of a compound of the invention for treating atrial fibrillation or symptoms thereof, is in a range of about 0.1 to about 100 mg/kg of a patient's body weight, more preferably in the range of about 0.5 to about 50 mg/kg of a patient's body weight, per daily dose. The compound can be administered for periods of short and long duration. Although some individual situations can warrant to the contrary, short-term administration, for example, 30 days or less, of doses larger than 25 mg/kg of a patient's body weight is preferred to long-term administration. When long-term administration, for example, months or years, is required, the suggested dose usually does not exceed 25 mg/kg of a patient's body weight.

Dosage ranges of pyridoxal-5′-phosphate typically are about 1 to about 1000 mg/day, about 100 to about 750 mg/day, about 100 to about 500 mg/day, about 100 to about 300 mg/day, about 200 to about 300 mg/day, about 200 to about 275 mg/day, about 225 to about 300 mg/day, about 225 to about 275 mg/day, about 230 to about 270 mg/day, about 240 to about 260 mg/day, and about 245 to about 255 mg/day. A dosage of about 250 mg/day of pyridoxal-5′-phosphate is also typical.

A therapeutically effective amount of a compound of the invention or a pharmaceutically acceptable addition salt of a compound of the invention for treating the above-identified diseases or symptoms thereof can be administered prior to, concurrently with, or after the onset of the disease or symptom. A compound of the invention can be administered concurrently. “Concurrent administration” and “concurrently administering” as used herein includes administering a compound of the invention and another therapeutic agent in admixture, such as, for example, in a pharmaceutical composition or in solution, or separately, such as, for example, separate pharmaceutical compositions or solutions administered consecutively, simultaneously, or at different times but not so distant in time such that the compound of the invention and the other therapeutic agent cannot interact and a lower dosage amount of the active ingredient cannot be administered.

In one embodiment of the invention, a method is provided for treating atrial fibrillation comprising administering a therapeutically effective amount of a compound of the invention or a pharmaceutically acceptable addition salt of a compound of the invention in a unit dosage form to a mammal. A method of treating atrial fibrillation can also include administering a therapeutically effective amount of a compound of the invention or a pharmaceutically acceptable addition salt of a compound of the invention in a unit dosage form to a mammal subsequent to a myocardial infarction or a cerebrovascular accident. A method of treating atrial fibrillation can also include administering a therapeutically effective amount of a compound of the invention or a pharmaceutically acceptable addition salt of a compound of the invention in a unit dosage form to a mammal following surgery including, but not limited to, invasive cardiovascular surgery, including, but not limited to coronary artery bypass graft (CABG), endarectomy, and heart valve replacement. Compounds of the invention or pharmaceutically acceptable salts thereof can be administered, alone or concurrently with other suitable therapeutic agents, following any angioplasty procedure. For instance, administration of said compounds may follow percutaneous transluminal angioplasty (PTA). PTA is used in coronary, pulmonary, peripheral, intracranial, extracranial carotid, renal, and aortic stenoses.

The compounds of the invention can also be used in combination with other therapeutic cardiovascular compounds that are generally used to treat cardiovascular or related diseases as well as symptoms thereof. A skilled physician or veterinarian readily determines a subject who is exhibiting symptoms of any one or more of the diseases described above and makes the determination about which compound is generally suitable for treating specific cardiovascular conditions and symptoms.

For example, atrial fibrillation can be treated by the administration of a compound of the invention or a pharmaceutically acceptable acid addition salt of a compound of the invention concurrently with another therapeutic agent. Other suitable therapeutic agents include, for example a angiotensin converting enzyme inhibitor, an angiotensin II receptor antagonist, a calcium channel blocker, an antithrombolytic agent, a β-adrenergic receptor antagonist, a diuretic, an α-adrenergic receptor antagonist, or a mixture thereof.

Methods of Treatment

A physician of ordinary skill can readily determine a subject who suffers from atrial fibrillation. Regardless of the route of administration selected, therapeutic compounds or a pharmaceutically acceptable salt thereof can be formulated into pharmaceutically acceptable unit dosage forms by conventional methods known to the pharmaceutical art. An effective but nontoxic quantity of the compound is employed in treatment.

A therapeutic compound or a pharmaceutically acceptable salt thereof can be administered in enteral unit dosage forms, such as, for example, tablets, sustained-release tablets, enteric coated tablets, capsules, sustained-release capsules, enteric coated capsules, pills, powders, granules, solutions, and the like. They can also be administered parenterally, such as, for example, subcutaneously, intramuscularly, intradermally, intramammarally, intravenously, and other administrative methods known in the art. They can further be administered nasally, sub-lingually, or in suppository form.

Although it is possible for a therapeutic compound or a pharmaceutically acceptable salt thereof as described above to be administered alone in a unit dosage form, preferably the compound is administered in admixture as a pharmaceutical composition.

The ordinarily skilled physician will readily determine and prescribe the therapeutically effective amount of a therapeutic compound or a pharmaceutically acceptable salt thereof to treat angina. In so proceeding, the physician could employ relatively low dosages at first, subsequently increasing the dose until a maximum response is obtained. Typically, the particular type of atrial fibrillation, the severity of the symptoms, or the frequency of the attacks, the compound to be administered, the route of administration, and the characteristics of the mammal to be treated, for example, age, sex, and weight, are considered in determining the effective amount to administer. In one embodiment of the invention, a therapeutic amount is in a range of about 0.1-100 mg/kg of a patient's body weight, more preferably in the range of about 0.5-50 mg/kg of a patient's body weight, per daily dose. The compound can be administered for periods of short and long duration. Although some individual situations can warrant to the contrary, short-term administration, for example, 30 days or less, of doses larger than 25 mg/kg of a patient's body weight is preferred to long-term administration. When long-term administration, for example, months or years, is required, the suggested dose should not exceed 25 mg kg of a patient's body weight.

A therapeutically effective amount of a therapeutic compound or a pharmaceutically acceptable salt thereof for treating atrial fibrillation can be administered prior to, concurrently with, or after the onset of atrial fibrillation.

A therapeutic compound of the invention can be administered concurrently with compounds that are already known to be suitable for treating atrial fibrillation. Concurrent administration” and “concurrently administering” as used herein includes administering a therapeutic compound and a known therapy in admixture such as, for example, in a pharmaceutical composition or in solution, or as separate components, such as, for example, separate pharmaceutical compositions or solutions administered consecutively, simultaneously, or at different times but not so distant in time such that the therapeutic compound and the known therapy cannot interact and a lower dosage amount of the active ingredient cannot be administered.

A compound suitable for use in methods of the invention and a therapeutic cardiovascular compound are administered concurrently. “Concurrent administration” and “concurrently administering” as used herein includes administering a compound suitable for use in methods of the invention and a therapeutic cardiovascular compound in admixture, such as, for example, in a pharmaceutical composition or in solution, or as separate compounds, such as, for example, separate pharmaceutical compositions or solutions administered consecutively, simultaneously, or at different times but not so distant in time such that the compound suitable for use in methods of the invention and the therapeutic cardiovascular compound cannot interact and a lower dosage amount of the active ingredient cannot be administered.

This invention will be further characterized by the following examples. These examples are not meant to limit the scope of the invention, which has been fully set forth in the foregoing description. Variations within the scope of the invention will be apparent to those skilled in the art.

### EXAMPLES

A randomized, double-blind placebo-controlled, dose-ranging, parallel-arm multi-center study was undertaken, on high-risk patients undergoing CABG surgery with cardiopulmonary bypass.

Patients were identified as “high risk” if they had two or more of the following risk factors:
Age greater than 65 years
Current smoker
History of diabetes mellitus requiring treatment other than diet
Evidence of left ventricular dysfunction or congestive heart failure
History of a previous non-disabling stroke, transient ischemic attack, or carotid endarterectomy
Urgent CABG intervention defined as the need to stay in the hospital (although the patient may be operated on within a normal scheduling routine).
History of myocardial infarction that occurred more than 48 hours but less than 6 weeks prior to CABG surgery
Prior peripheral artery surgery or angioplasty
Moderate renal dysfunction defined as creatine >133 μmol/L (1.5 mg/dl), but <250 μmol/L (2.8 mg/dl)
Presence of at least one asymptomatic carotid artery stenosis (>50% ) either in one or two carotid arteries.

Approximately 900 high risk pre-CABG patients in 42 different treatment centers in North America were screened and randomized to three groups of approximately 300 patients each, prior to their bypass surgery, as follows.

Patients were either placed in a control group (placebo), treated with 250 mg/day of pyridoxal-5′-phosphate, (250 mg/day), or treated with 750 mg day of pyridoxal-5′-phosphate (750 mg/day). The first dose of study medication was administered at 3-10 hours prior to CABG surgery. In the event of surgery delay or rescheduling, a second pre-operative dose of pyridoxal-5′-phosphate was administered so that all patients received study medication 3-10 hours before surgery. Treatment continued for 30 days after surgery (post operative day (POD) 30). Patients received follow-up evaluations up to and including postoperative day (POD) 4, on POD 30 and on POD 90.

Patients were measured for combined incidence of cardiovascular death, nonfatal myocardial infarction (MI), and nonfatal cerebral infarction up to and including post-operative day 30 (POD 30). Patients were also measured for nonfatal myocardial infarction alone. All deaths without an identifiable non-cardiovascular cause were considered of cardiovascular origin.

Cerebral infarction (stroke) was defined clinically as any new sudden onset focal neurological deficit lasting at least 24 hours as assessed by a neurologist and after neuroimaging (computed tomography [CT] Brain Scan or magnetic resonance imaging [MRI]) has excluded an intracerebral hemorrhage. All patients suspected clinically of having a stroke or transient ischemic attack (TIA) received a neurological examination (conducted by a neurologist or internist with expertise in cerebral vascular disease) within 24 hours of onset of symptoms. All patients suspected clinically of having a stroke were submitted to cerebral imaging. When determining whether a patient had myocardial infarction, the following definitions were used:
A peak creatine kinase—myocardial band (CK-MB) above a certain threshold. Since different experts and different prior art clinical trials used different cut-offs for this threshold, three different thresholds were used to determine whether a patient had myocardial infarction. For Experiment 1A, the determination of whether the patient had myocardial infarction was made using a cut off threshold of 100 ng/ml. Thus, in Experiment 1A, patients with peak CK-MB of 100 ng/ml or greater on days up to and including POD 4 were considered to have myocardial infarction. For Experiment 1B, a cut off threshold of 70 ng/ml was used. For Experiment 1C, a cut off threshold of 50 ng/ml was used;
A new q-wave evidence of myocardial infarction along with CK-MB of 35 ng/ml or above on days up to and including POD 4;
A peak CK-MB of 5× ULN (25 ng/ml) or above occurring after POD 4;
A new q-wave evidence of myocardial infarction that was not present at POD 4; or

A q-wave or non-q-wave myocardial infarction as identified by the investigator and confirmed by the Clinical Endpoint Committee.

TABLE 1
Timetable of Visits and Procedures

| | Assessment Period | | | | | | | | | | | |
|---|---|---|---|---|---|---|---|---|---|---|---|---|
| | Pre-Op | Post-Op Double-Blind Treatment Period | | | | | | | | | | Follow-up |
| | Post-Op Days (POD) | | | | | | | | | | | |
| | | | | | | 1 | | 2 | 3 | 4 | 30 | 90 |
| | Hours Post-CABG | | | | | | | | | | | |
| | | 4 | 8 | 12 | 16 | 24 | 36 | 48 | 72 | 96 | | |
| Informed consent | X | | | | | | | | | | | |
| Inclusion/exclusion | X | | | | | | | | | | | |
| Medical/surgical history | X | | | | | | | | | | | |
| Physical exam | X | | | | | | | | | | X | X |
| Vital signs | X | | | | | | | X | | X | X | X |
| 12-lead ECG | X | | | | | | | X | | X | X | X |
| NIH Stroke Scale | X | | | | | | | | | As | As | As |
| | | | | | | | | | | needed | needed | needed |
| Psychometric testing | optional | | | | | | | | | optional | optional | optional |
| Modified Rankin Scale | X | | | | | | | | | As | X | X |
| | | | | | | | | | | needed | | |
| MMSE | X | | | | | | | | | | X | X |
| Chemistry and hematology | X | | | | | | | | | X | X | X |
| CK-MB | X | X | X | X | X | X | X | X | X | X | X | X |
| Ancillary tests * | X | | | | | | | | | | X | X |
| Record AEs | X | X | X | X | X | X | X | X | X | X | X | X |
| Review concomitant meds | X | X | X | X | X | X | X | X | X | X | X | X |
| Administer/Dispense | X | | | | | | | | | X | | |
| medication | | | | | | | | | | | | |

Results-Atrial Fibrillation

12-lead ECG were taken at baseline (Pre-Op), POD 2, POD 4, POD 30 and POD 90 with patients on placebo, 250 mg/day pyridoxal-5′-phosphate (P5P), and 750 mg/day pyridoxal-5′-phosphate.

The data from baseline, POD 4 and POD 90 were analyzed.

TABLE 2
Number of patients who had AF at baseline, by treatment group:

| | Placebo | 250 mg/day | 750 |
|---|---|---|---|
| | (n = 299) | P5P (n = 301) | mg/day P5P (n = 302) |
| Atrial Fibrillation | 15 (5.02%) | 18 (5.98%) | 14 (4.64%) |

TABLE 3
Number of patients who had AF at POD 4, by treatment group:

| | Placebo | 250 mg/day | 750 |
|---|---|---|---|
| | (n = 299) | P5P (n = 301) | mg/day P5P (n = 302) |
| Atrial Fibrillation | 54 (18.06%) | 56 (18.60%) | 63 (20.86%) |

TABLE 4
Number of patient who had AF at POD 90, by treatment group:

| | Placebo | 250 mg/day | 750 |
|---|---|---|---|
| | (n = 299) | P5P (n = 301) | mg/day P5P (n = 302) |
| Atrial Fibrillation | 35 (11.70%) | 17 (5.65%) | 18 (5.96%) |

Compared to placebo, which had 11.9% of patients with AF, the 250 mg pyridoxal-5′-phosphate dose reduced AF by 51.75% (p=0.0084), and the 750 mg pyridoxal-5′-phosphate dose reduced it by 49.08% (p=0.013). These results were observed from post operative day 4 to post operative day 90.

What is claimed is:

1. A method of treating atrial fibrillation in a mammal comprising: administering to the mammal a therapeutically effective amount for treating atrial fibrillation of a pharmaceutical composition comprising pyridoxal-5′-phosphate, pyridoxal, pyridoxamine, 3-acylated analogues of pyridoxal, 3-acylated analogues of pyridoxal-4,5-aminal, pharmaceutically acceptable acid salts, and mixtures thereof.

2. A method of claim 1, wherein the atrial fibrillation is subsequent to myocardial infarction.

3. A method of claim 1, wherein the atrial fibrillation occurs post-cardiac surgery.

4. A method of claim 3, wherein the surgery is selected from the group consisting of bypass surgery, thrombolysis, and angioplasty.

5. A method of claim 4, wherein the bypass surgery is coronary artery bypass grafting.

6. The method of claim 1, wherein the mammal is human.

7. The method of claim 1, wherein the therapeutic amount is about 100 mg per day to about 1000 mg per day.

8. The method of claim 1, wherein the therapeutic amount is about 200 mg per day to about 300 mg per day.

9. The method of claim 1, wherein the therapeutic amount is about 250 mg per day.

10. The method of claim 1, wherein the therapeutic amount is about 750 mg per day.

11. The method of claim 1, wherein said compound is administered enterally or parenterally.

12. The method of claim 1, wherein said compound is pyridoxal-5′-phosphate or a pharmaceutically acceptable salt thereof.

13. The method of claim 1, wherein said compound is pyridoxal or a pharmaceutically acceptable salt thereof.

14. The method of claim 1, wherein the said compound is pyridoxamine or pharmaceutically acceptable salt thereof.
